(19) United States
(12) Patent Application Publication
(10) Pub. No.: US 2012/0093782 A1
(43) Pub. Date: Apr. 19, 2012

(54) Enhanced Hematopoietic Stem Cell Engraftment

(76) Inventors: Robert I. Grove, Avon, IN (US); Paul A. Hyslop, Indianapolis, IN (US)

(21) Appl. No.: 13/262,972

(22) PCT Filed: May 8, 2010
(86) PCT No.: PCT/US2010/034154

§ 371 Date: Oct. 5, 2011

Related U.S. Application Data
(60) Provisional application No. 61/177,835, filed on May 13, 2009.

(51) Int. Cl. A61K 35/12; A61P 35/02; A61P 7/06; A61P 7/00

(52) U.S. Cl. 424/93.7

(57) ABSTRACT

The invention relates to improved products, processes, and therapeutic methods relating to hematopoietic stem cells and hematopoietic stem cell transplantation. Included are methods for improving transplant efficiency of cord blood units comprising use of mixtures of expanded CD34⁺/CD 133⁻ HSCs and unexpanded CD133⁺ HSCs for IBM administration.

This application claims priority from U.S. Provisional Patent Application Ser. No. 61/177,835 the entire contents of which is herein incorporated by reference.

### TECHNICAL FIELD

The present invention relates generally to products, methods and processes pertaining to hematopoietic stem cells (HSCs) for therapeutic use in mammals including stem cell transplantation for hematopoietic reconstitution.

### BACKGROUND OF THE INVENTION

Hematopoietic stem cells (HSC) are pluripotent stem cells characterized by their ability to give rise under permissive conditions to all cell types of the hematopoietic system. The frequency of HSCs in bone marrow is very low (e.g. one HSC/10⁵ bone marrow cells in mice; Harrison D. E. et al., Exp. Hematol. 21, 206-219, (1993). Marker phenotypes useful for identifying HSCs will be those commonly known in the art. For human HSCs, the cell marker phenotypes preferably include CD34⁺CD38⁻CD90 (Thy1)⁺ Lin⁻. For mouse HSCs, an exemplary cell marker phenotype is Sca⁻1¹CD90¹ (see, e.g., Spangrude, G. J. et al., Science 1:661-673 (1988)) or c-kit¹ Thy1^{1o} Lin⁻ Sca-1⁺ (see, Uchida, N. et al., J. Clin. Invest. 101(5):961-966 (1998)). Alternative HSC markers such as aldehyde dehydrogenase (see Storms et al., Proc. Nat'l Acad. Sci. 96:9118-23 (1999) and AC133 (see Yin et al., Blood 90:5002-12 (1997) are also useful.

Hematopoietic progenitor cells (HPCs) are undifferentiated cell types that exhibit the highest proliferation potential of all other cells, and are capable of producing one or more lineage-specific, differentiated cell types. HPCs typically follow several differentiation pathways to form the complete repertoire of mature cells found circulating in adult blood.

Biological systems such as the hematopoietic system exhibit a so-called “stem cell hierarchy” in which stem cells are designated “primitive” or “mature” based on cell cycle and a number of other parameters. Primitive stem cells are quiescent, while a greater proportion of mature stem cells are in an active cell cycle. Primitive stem cells have a greater proliferation potential than mature stem cells. As primitive stem cells become mature, their self-renewal capacity, proliferation potential, and “sternness” decreases which means that stem cell potency also decreases. When a stem cell enters a specific lineage pathway and becomes determined, the potency effectively drops to zero.

Transplantation of allogeneic or autologous hematopoietic stem cells is an established treatment for a variety of diseases including hematological cancers, metabolic disorders, and certain genetic diseases. Intravenous (IV) injection is currently the preferred method for transplanting hematopoietic cells. Transplanted hematopoietic stem cells home to bone marrow, where they find their “niches” and seed. Bone marrow niches provide the necessary environment to allow HSCs to divide either symmetrically to produce two daughter HSCs, or two daughter hematopoietic progenitor cells (HPCs), or asymmetrically to produce both HSC and HPC daughter cells. The self-renewal potential of HSCs provides a continuous supply of undifferentiated stem cells for replentishment of the hematopoietic system, and ensures that all bone marrow compartments contain HSCs. The capacity for self-renewal also enables intra-bone marrow (IBM) grafting wherein IBM grafts placed in a single marrow compartment can replenish the entire system.

From syngeneic murine studies, it is known that around 10% of infused hematopoietic stem cells home to bone marrow after a conventional IV injection. Cashman J. D. and Eaves, C. J., Blood, 96, 3979-3981 (2000). The majority of HSCs administered IV become sequestered in other organs such as lung and liver. Szilvassy, S J, et al., Blood, 93, 1557-1566, (1999). It has been determined that bone marrow seeding efficiency improves about 15-fold when myeloablated mice are administered murine bone marrow cells by the intra-bone marrow (IBM) route instead of IV administration. Castello, S. et al., Exp. Hematol., 32, 782-787, (2004).

Sources of allogeneic HSCs for transplantation include HSCs from donor bone marrow in which HSCs have been mobilized using GCSF, and cord blood derived HSCs. Cord blood (“CB”) HSCs have several distinct advantages over BM derived HSCs. First, only four out of six alleles are required to match at the three major histocompatability antigens relevant to engraftment and minimal GvH or HvG responses (i.e. HLA-A,-B and HLA-DRB1). By contrast, BM-derived HSC transplants require a match at all six alleles. As a result, donor-recipient matching for BM-derived HSC transplant represents a significant challenge to finding a suitable donor in the general population. Second, cord blood HSCs are readily available and can be harvested and banked at any time. Third there is increasing evidence that a 2/6 HLA mismatch may have a positive effect in graft versus leukemia response. And fourth, CB transplants have a low risk of transmissible infectious diseases.

On the other hand, a disadvantage associated with cord blood HSC transplants is that the count of HSCs in the CB unit may be lower than the threshold needed for successful engraftment, particularly in adults. This can be a significant hurdle. Many units of CB collected under the AABMT approved protocols fail to provide an adequate total nucleated cell count (TNC). For Caucasians, the required TNC is ≧1.1×10⁹. For other racial groups, such as Blacks or Hispanics, the qualifying TNC count is relaxed somewhat to ≧0.8×10⁹ to account for a lower chance of finding a match in such groups, fewer absolute numbers of donors, and fewer donations that meet the TNC cutoff applied to Caucasians.

Other challenges associated with HSC transplants and in particular CB transplants include the absence of reliable assays to accurately assess the number of self-renewing HSCs in the unit that will engraft, the rate of engraftment and the quality of the cells being transplanted. These and other shortcomings associated with cord blood transplants have translated into increased graft failure, and perhaps more importantly, increased time for a recipient's endogenous immune system to become functional and platelet count to rise to an acceptable level. During post-procedure recovery, transplant patients must be confined to an intensive care unit. Per diem costs for post transplant conditioning regimens vary greatly, and will depend on variables that include costs of the patient's required medications, health and age, and fixed overhead costs for a given transplant center. Major cost savings can be accomplished by reducing the length of patient stay by 8.6 days resulting in cost reduction per case of up to $100,000, or approximately $12,000 per day. (Hospital Case management: the monthly update on hospital-based care planning and critical paths. In Hosp Case Manag. (1998) 3 43-6). Given that a typical transplant unit of HSCs costs about $35,000 it is evident that BM transplantation and post-transplant care creates a significant financial burden on the healthcare system.

There remains a need for products, methods and processes that improve HSC transplantation efficiency, including enhanced engraftment potential for cord blood transplants. The present invention pertains to products, methods, processes, and compositions that relate to (1) improved methods of treatment for patients suffering from impaired hematopoiesis that enhance engraftment potential and myeloid replacement thereby minimizing time in the ICU, and enabling use of CB units that fall below the currently acceptable TNC.

### SUMMARY OF THE INVENTION

It is an object of the present invention to enhance the transplant potential and engraftment of hematopoietic stem cells for improved therapeutic applications including but not limited to cord blood transplantations.

It is another object of the present invention to provide improved methods, processes, products, kits and compositions for improving hematologic transplantation potential and reconstitution in a transplant patient.

These and other objects of the invention are evidenced by the summary of the invention, the description of the various embodiments, and the claims.

In one aspect, the present invention relates to therapeutic products including compositions and/or kits comprising expanded and/or unexpanded HSCs for reconstituting hematopoiesis in a mammalian host. In one embodiment, the HSCs are derived from a human source wherein the expanded HSCs are CD34⁺/CD133⁻ and the unexpanded HSCs are CD133⁺.

In another aspect, the present invention relates to methods and processes for enhancing HSC transplant potential and efficiency by isolating and admixing purified expanded and purified unexpanded HSCs wherein the expanded cells are CD34⁺/CD133⁻ and the unexpanded cells are CD133⁺ such that the ratio of expanded cells to unexpanded cells is in a range of about 90:10 to about 70:30.

In another aspect, the present invention relates to methods for treating a patient suffering from impaired hematopoiesis by administering a mixture of purified expanded CD34⁺ and unexpanded CD133 HSCs.

### BRIEF DESCRIPTION OF THE DRAWINGS

FIG. 1. ATP-based assay for HSC potency.

FIG. 2. ATP standard curves for CD133⁺ HSC GEMM and CD133⁺ HPP cells.

FIG. 3. Methylcellulose colony forming assay on expanded versus unexpanded cells.

FIG. 4. Percent chimerism in NOD/SCID mouse BM following IV or intra-BM administration of a 20:80 mixture of CD133⁺/CD34⁺ CD133⁻ human cord blood cells.

FIG. 5. Percent human CD45⁺ cells in peripheral blood of NOD/SCID mice following intra-BM injection of CD133¹/CD34¹ CD133⁻) from human umbilical cord blood.

FIG. 6. Serial transplant efficiency of expanded CD34⁺ cells.

### DETAILED DESCRIPTION

In reference to the present disclosure, the technical and scientific terms used in the descriptions herein will have the meanings commonly understood by one of ordinary skill in the art, unless specifically defined otherwise. Accordingly, the following terms are intended to have the following meanings.

“Allogeneic” refers to deriving from, originating in, or being members of the same species, where the members are genetically related or genetically unrelated but genetically similar. An “allogeneic transplant” refers to transfer of cells or organs from a donor to a recipient, where the recipient is the same species as the donor.

“Autologous” refers to deriving from or originating in the same subject or patient. An “autologous transplant” refers to the harvesting and reinfusion or transplant of a subject's own cells or organs. Exclusive or supplemental use of autologous cells can eliminate or reduce many adverse effects of administration of the cells back to the host, in particular graft versus host reaction.

“Committed myeloid progenitor cell” or “myeloid progenitor cell” refers to a multipotent or unipotent progenitor cell capable of ultimately developing into any of the terminally differentiated cells of the myeloid lineage, but which do not typically differentiate into cells of the lymphoid lineage. Hence, “myeloid progenitor cell” refers to any progenitor cell in the myeloid lineage. Committed progenitor cells of the myeloid lineage include oligopotent CMP, GMP, and MEP as defined herein, but also encompass unipotent erythroid progenitor, megakaryocyte progenitor, granulocyte progenitor, and macrophage progenitor cells. Different cell populations of myeloid progenitor cells are distinguishable from other cells by their differentiation potential, and the presence of a characteristic set of cell markers.

“Cytokine” refers to compounds or compositions that in the natural state are made by cells and affect physiological states of the cells that produce the cytokine (i.e., autocrine factors) or other cells. Cytokine also encompasses any compounds or compositions made by recombinant or synthetic processes, where the products of those processes have identical or similar structure and biological activity as the naturally occurring forms. Lymphokines refer to natural, synthetic, or recombinant forms of cytokines naturally produced by lymphocytes, including, but not limited to, IL-1, IL-3, IL-4, IL-6, IL-11, and the like.

“Expansion” refers to increase in the number of a characteristic cell type, or cell types, from an initial population of cells, which may or may not be identical. The initial cells used for expansion need not be the same as the cells generated from expansion. For instance, the expanded cells may be produced by growth and differentiation of the initial population of cells.

“Functional” in the context of cells refers to cells capable of performing or cells that retain the regular functions or activities associated with the specified cell type, as identified by a defined functional assay or assays. For instance, a “functional GMP cell” is a progenitor cell capable of ultimately differentiating into granulocytes and macrophages, where the terminally differentiated cells function as normal granulocytes and macrophages.

“Graft-versus-host response” or “GVH” or “GVHD” refers to a cellular response that occurs when lymphocytes of a different MHC class are introduced into a host, resulting in the reaction of the donor lymphocytes against the host.

“Granulocyte/macrophage progenitor cell” or “GMP” refers to a cell derived from common myeloid progenitor cells, and characterized by its capacity to give rise to granulocyte and macrophage cells, but which does not typically give rise to erythroid cells or megakaryocytes of the myeloid lineage.

“Growth factor” refers to a compound or composition that in the natural state affects cell proliferation, cell survival, and/or differentiation. A growth factor may also affect other physiological process, such as secretion, adhesion, response to external stimuli, and the like. Although many growth factors are made by cells, growth factors as used herein also encompass any compound or composition made by recombinant or synthetic processes, where the product of those processes have identical or similar structure and biological activity as the naturally occurring growth factor. Examples of growth factors include but are not limited to epidermal growth factor (EGF), fibroblast growth factor (FGF), erythropoietin (EPO), thromobopoietin (TPO), stem cell factor (SCF), and flt-3 ligand (FL), and analogs thereof.

“Isolated” refers to a product, compound, or composition which is separated from at least one other product, compound, or composition with which it is associated in its naturally occurring state, whether in nature or as made synthetically.

“Hematopoietic stem cell” or “HSC” refers to a clonogenic, self-renewing pluripotent cell capable of ultimately differentiating into all cell types of the hematopoietic system, including B cells, T cells, NK cells, lymphoid dendritic cells, myeloid dendritic cells, granulocytes, macrophages, megakaryocytes, and erythroid cells. As with other cells of the hematopoietic system, HSCs are typically defined by the presence of a characteristic set of cell markers. “Enriched” when used in the context of HSC refers to a cell population selected based on the presence of a single cell marker, e.g. CD34⁺, while “purified” in the context of HSC refers to a cell population resulting from a selection on the basis of two or more markers, preferably CD34⁺CD90+.

“Marker phenotyping” refers to identification of markers or antigens on cells for determining their phenotype (e.g., differentiation state and/or cell type). This may be done by immunophenotyping, which uses antibodies that recognize antigens present on a cell. The antibodies may be monoclonal or polyclonal, but are generally chosen to have minimal crossreactivity with other cell markers. It is to be understood that certain cell differentiation or cell surface markers are unique to the animal species from which the cells are derived, while other cell markers will be common between species. These markers defining equivalent cell types between species are given the same marker identification even though there are species differences in structure (e.g., amino acid sequence). Cell markers include cell surfaces molecules, also referred to in certain situations as cell differentiation (CD) markers, and gene expression markers. The gene expression markers are those sets of expressed genes indicative and/or characteristic of the cell type or differentiation state. In part, the gene expression profile will reflect the cell surface markers, although they may include non-cell surface molecules.

“Mismatched allogeneic” refers to deriving from, originating in, or being members of the same species having non-identical major histocompatability complex (MHC) antigens as typically determined by standard assays used in the art, such as serological or molecular analysis of a defined number of MHC antigens. A “partial mismatch” refers to partial match of the MHC antigens tested between members, typically between a donor and recipient. For instance, a “half mismatch” refers to 50% of the MHC antigens tested as showing different MHC antigen type between two members. A “full” or “complete” mismatch refers to all MHC antigens tested as being different between two members.

“Myeloablative” or “myeloablation” refers to impairment or destruction of the hematopoietic system, typically by exposure to a cytotoxic agent or radiation. Myeloablation encompasses complete myeloablation brought on by high doses of cytotoxic agent or total body irradiation that destroys the hematopoietic system. It also includes a less than complete myeloablated state caused by non-myeloablative conditioning. Thus, non-myeloablative conditioning is treatment that does not completely destroy the subject's hematopoietic system.

“Neutropenia” refers to a lower than normal number of neutrophils and other polymorphonuclear leukocytes in the peripheral blood. Typically, a neutropenic condition is diagnosed based on the absolute neutrophil count (ANC), which is determined by multiplying the percentage of bands and neutrophils on a differential by the total white blood cell count. Clinically, an abnormal ANC is fewer than about 1500 cells per ml of peripheral blood. The severity of neutropenia is categorized as mild for an ANC of 1000-1500 cells per ml, moderate for an ANC of 500-1000 cells per ml, and severe for an ANC of fewer than 500 cells per ml.

“Self renewal” refers to the ability of a cell to divide and generate at least one daughter cell with the identical (e.g., self-renewing) characteristics of the parent cell. The second daughter cell may commit to a particular differentiation pathway. For example, a self-renewing hematopoietic stem cell divides and forms one daughter stem cell and another daughter cell committed to differentiation in the myeloid or lymphoid pathway. A committed progenitor cell has typically lost the self-renewal capacity, and upon cell division produces two daughter cells that display a more differentiated (i.e., restricted) phenotype.

The term “proliferation” refers to the expansion of HSCs by continuous division into initially two identical daughter cells. Proliferation occurs prior to differentiation.

The term “stem cell potency” as used herein relates to any of the following properties of transplanted hematopoietic stem cells (HSC) bearing the CD133 marker isolated from cord blood MNC's: (1) The number of CD133⁺ HSC in a transplanted unit that have the potential of life-long ability to self renew and proliferate their numbers by symmetric division to yield identical daughter HSC cells within the bone marrow niche; (2) the numbers of CD133¹ HSC in a transplanted unit that have the potential to be able to self-renew and have the potential life-long ability to maintain their numbers by asymmetric division to yield an identical HSC and a committed hematopoietic daughter progenitor cell (HPC); (3) the number of CD133⁺ HSC that have the potential ability to be able to exit the bone marrow compartment where they are transplanted, and have the ability to “home’ to other non-contiguous bone marrow compartments, such that life-long hematopoiesis is distributed to other non-contiguous bone marrow compartments other than the transplant site.

The term “progenitor cell potency” as used herein relates to the following properties of hematopoietic progenitor cells (HPC) that have been derived from an in vitro proliferated population of cells originally bearing the CD 34 marker isolated from cord blood MNC's in vitro. In a standardized colony-forming assay, the numbers of progenitor colonies counted that are obtained from a given number of proliferated cells seeded into the colony forming assay as shown in Table 1.

Potency of HSCs and HPCs may be determined by any suitable method that measures, for example, the potency of unexpanded CD133⁺ HSCs may be determined by measuring intracellular ATP concentrations against a suitable standard. A suitable assay for this purpose has been developed by HemoGenix, Inc.

TABLE 1
Methyl Cellulose CFU Progenitor Cell Assay

| 550 CD34⁺/CD34⁻ | BFU-E | CFU-GM | CFU-GEMM | |
|---|---|---|---|---|
| cells/dish | (%) | (%) | (%) | Colonies |
| Freshly Isolated | 32 | 59 | 10 | 169 |
| Expanded (~160 | 39 | 49 | 6 | 93 |
| fold) | | | | |

The term “Substantially pure cell population” or “purified” as applied to cells refers to a population of cells having a specified cell marker characteristic and differentiation potential that is at least about 50%, preferably at least about 75-80%, more preferably at least about 85-90%, and most preferably at least about 95% of the cells making up the total cell population. Thus, a “substantially pure cell population” refers to a population of cells that contain fewer than about 50%, preferably fewer than about 20-25%, more preferably fewer than about 10-15%, and most preferably fewer than about 5% of cells that do not display a specified marker characteristic and differentiation potential under designated assay conditions.

“Subject” or “patient” are used interchangeably and refer to, except where indicated, mammals such as humans and non-human primates, as well as rabbits, rats, mice, goats, pigs, and other mammalian species.

“Syngeneic” refers to deriving from, originating in, or being members of the same species that are genetically identical, particularly with respect to antigens or immunological reactions. These include identical twins having matching MHC types. Thus, a “syngeneic transplant” refers to transfer of cells or organs from a donor to a recipient who is genetically identical to the donor.

As used herein “transplant potential” relates to the likelihood that a HSC or population thereof will result in a successful transplant.

“Thrombocytopenia” refers to a lower than normal platelet count, generally less than about 100×10⁹/L, which gives rise to increased clotting time and increased risk of spontaneous bleeding, particularly at platelet levels of about 10-50×10⁹/L or lower. The condition occurs when platelets are lost from circulation at a faster rate than their replenishment by megakaryocytes. Thrombocytopenia may result from either failure of platelet synthesis and/or increased rate of platelet destruction.

“Xenogeneic” refers to deriving from, originating in, or being members of different species, e.g., human and rodent, human and swine, human and chimpanzee, etc. A “xenogeneic transplant” refers to transfer of cells or organs from a donor to a recipient where the recipient is a species different from that of the donor.

The present invention relates in one aspect to processes, methods, and compositions for improving engraftment and reconstitution of the hematopoietic system following transplantation of HSCs. In one aspect, the invention relates to a mixture of expanded and unexpanded cell populations purified from any suitable source of HSCs including peripheral blood, bone marrow, cord blood and other sources known to contain hematopoietic progenitor cells including, for example, liver or fetal liver. Preferably the source of HSCs is human cord blood. The rationale for using expanded stem cells is that in vitro expansion leads to differentiation of mature cells including neutrophils and platelets thereby enabling shortened recovery times after transplantation.

The expanded cell component of the mixture is enriched for stem and/or progenitor cells having the CD34⁻ cell surface marker. Cells are expanded from 5-fold to 500-fold or more in vitro by any suitable method, for example, by the methods disclosed in US 2002/0132343 and US 2006/0134783, the entire contents of which are herein incorporated by reference. Purified CD34⁺ cells from an ex-vivo expanded population are preferably frozen and stored under suitable cryopreservation conditions known in the art, for example, as disclosed in US 2006/0134783.

The growth and differentiation potential and/or potency of the expanded CD34⁺ cells is determined by any suitable method, for example the methylcellulose colony forming assay. The CFC assay allows, where appropriate, detection of total colony forming units including CFU-GM, BFU-E, CFU-HPP, and CFU-GEMM.

Admixtures of HSCs of the invention also include unexpanded cells that are enriched for CD133⁺. While the purpose for the expanded CD34⁺ population is to have sufficient mature cells for reconstitution of the hematopoietic system in order to reduce time to recovery, the purpose for the unexpanded CD133⁺ population is to provide an adequate source of more primitive stem cells.

As previously mentioned, a challenge associated with transplantation, and in particular transplantations based on CB sources, is that the stem cell count in a CB unit often is below threshold standards needed for a successful transplant. Moreover, there currently is no nationally or internationally-recognized, validated and accepted assay to determine HSC potency.

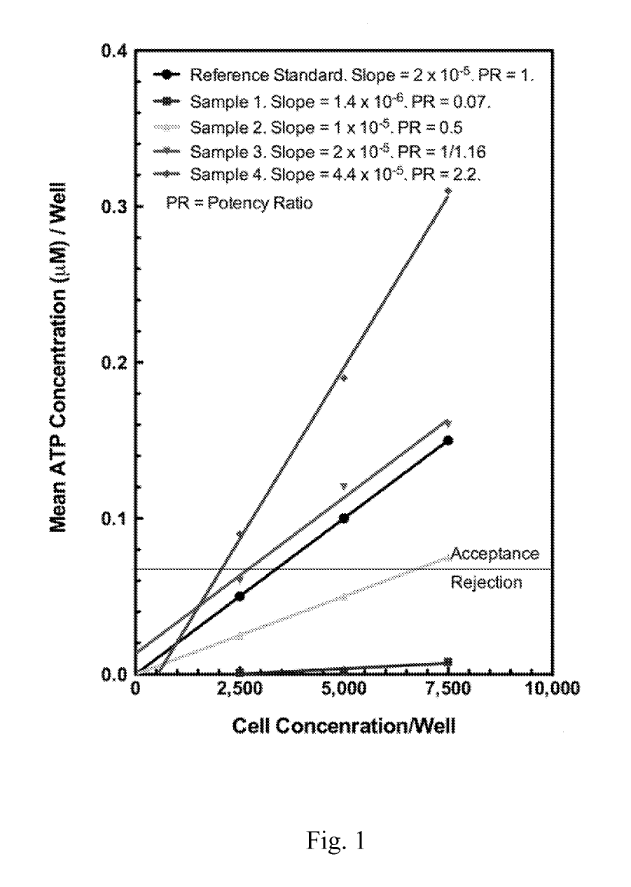

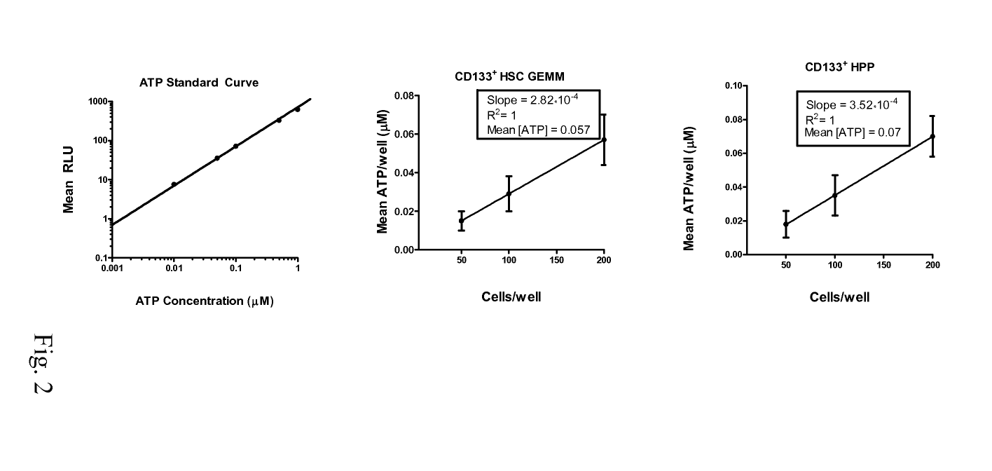

In one embodiment, the invention relates to admixing unexpanded CD133⁺ cells with expanded CD34⁺. Preferably, the purified CD133⁺ fraction is pre-tested for potency prior to use to determine potential for engraftment. While potency can be determined by any suitable means, a preferred method relies on measuring intracellular ATP (i-ATP) concentrations against a suitable standard (see FIGS. 1-2). Thus, in one aspect the invention relates to admixing expanded CD34¹ and unexpanded CD133⁺ cells which have undergone a suitable potency test prior to or after admixing and administration.

Cell compositions and/or admixtures of the invention comprise expanded CD34¹/CD133⁻ and unexpanded CD133⁺ cells in a ratio of about 70:30; preferably about 80:20; most preferably about 90:10.

Cryopreservation of Expanded CD34⁺/CD133⁻ Cells

The expanded population of cells described herein can be cryopreserved and stored for future use and still retain their functionality. A variety of mediums and protocols for freezing cells are known in the art. Generally the cells are concentrated, suspended in a medium supplemented with a cryoprotectant and/or stabilizer, frozen and stored at a temperature of 0° C. or less. In some embodiments the cells are stored at −70° C. or less, e.g. −80° C., or in liquid nitrogen or in the vapor phase of liquid nitrogen. The cells can be stored in any cryoprotectant known in the art. For example, the cryoprotectant can be dimethyl sulfoxide (DMSO) or glycerol. In some embodiments, the freezing medium comprises DMSO from about 5-10%, 10-90% serum albumin, and 50-90% culture medium. In some embodiments, the cryopreservation medium will comprise DMSO about 7.5%, about 42.5% serum albumin, and about 50% culture medium. The cells can be stored in any stabilizer known in the art. For example, the stabilizer may be methyl cellulose or serum.

Prior to freezing, the cells may be portioned into several separate containers to create a cell bank. The cells may be stored, for example, in a glass or plastic vial or tube or a bag. When the cells are needed for future use, a portion of the cryopreserved cells (from one or more containers) may be selected from the cell bank, thawed and used.

Human CD45 Analysis for Pre-clinical Animal Studies

After red cell reduction and counting, preclinical samples are labeled with anti-human CD45 antibody (Pharmingen) and analyzed by flow cytometry. At least 1×10⁶ cells are stained per tube for 10 minutes at 4° C., washed with DPBS+0.5% BSA+2mMEDTA and centrifuged for 10 minutes at 4° C. and 200×g. Total percent human CD45⁺ is determined by subtracting the Isotype control from the total human CD45⁺.

### CD133⁺ Fraction Potency Assay

HALO-96 PQR (Hemogenix) is an ATP bioluminescence proliferation assay to measure stem cell potency and define acceptable limits for transplantation. The assay utilizes a reference standard that allows the potency ratio of a sample to be quantified. HALO®-96 PQR complies with the requirements of the Standards Organizations, as well as the guideline requirements of the FDA and EMEA. HALO®-96 PQR is based on the direct correlation of the intracellular ATP (iATP) concentration with proliferation potential, viability, cell number and cellular and mitochondrial integrity.

Cells from each of 3 cell doses are added to individual tubes containing a HALO® Master Mix with growth factors that stimulate multipotential stem cells, i.e. CFC-GEMM. Inclusion of the more primitive stem cell population, HPP-SP, also adds greater reliability to the potency measurement. Freshly prepared cord blood CD133⁺ cells and a similar cell dose response prepared and added to tubes containing HALO® Master Mix. After mixing the cells with the Master Mix, 6 replicates of the HALO® Culture Master Mix are dispensed into the wells of a 96-well plate. The cells are incubated for 5 days. Prior to processing the sample plate, an ATP standard curve is generated. After incubation, the cells are mixed with a cell lysis reagent and luciferin/luciferase. The released iATP acts as a limiting substrate for the luciferin/luciferase reaction to produce bioluminescence, which can be measured in a plate luminometer. The slope of the cell dose response provides the basis to determine the potency ratio. Incorporating an ATP external standard in the assay allows direct comparison of reference standard(s) and samples.

The iATP assay allows assessment of the proliferation potential and potency of the stem cells which correlates with the probability that stem cells will engraft. Potency is measured as the slope of mean ATP/well (uM) against number cells/well (See FIG. 2). Acceptable potency levels can be determined with adequate testing; a provisional level set by HemoGenix is about 1×10⁻⁵.

Examplary iATP Assay

Four sets of cell suspensions are counted using a Beckman Coulter Z2 particle counter and the cell concentrations for each cell suspension adjusted to a working cell concentration of 2×10⁴/ml in IMDM. The viability of each cell suspension is also determined using the 7-aminoactinomycin D (7-AAD) dye exclusion method for flow cytometry. Labeling is performed according to instructions from Beckman-Coulter.
(1) The 2×10⁴/ml working dilution gives a final dilution of 200 cells/well when 0.1 ml to 0.9 ml of HALO Master Mix.
(2) The 2×10⁴/ml working dilution is diluted to 1×10⁴/ml, which in turn is further diluted to 0.5×10⁴/ml. 0.1 ml of each working dilution is added to 0.9 ml of HALO Master Mix. The final concentration per well is 100 cells/well and 50 cells/well respectively.
(3) A 3-point cell dose response of 200, 100 and 50 cells/well is performed for each of the 4 cell suspensions.
(4) Each cell working dilution from each cell suspension is added to a HALO-96 MeC Master Mix for:
(5 Background control containing no growth factors.
(6) HPP-SP (High Proliferative Potential-Stem and Progenitor Cells) containing EPO, GM-CSF, G-CSF, IL-3, IL-6, SCF, TPO, Flt3-L, IL-2 and IL-7
(7) CFC-GEMM containing EPO, GM-CSF, G-CSF, IL-3, IL-6, SCF, TPO, Flt3-L.
(8) In addition to the growth factors and cytokines, the HALO Master Mix contained methyl cellulose and 5% fetal bovine serum.
(9) Each serial cell dose response for each set of cell populations to be tested from each cell suspension is set up in a separate 96-well, white-walled culture plate. Eight replicates each of 0.1 ml are dispensed into each column of wells using an electronic repeater pipette (Eppendorf). The cells are incubated for 7 days at 37° C. in a fully humidified incubator containing 5% CO₂ and 5% O₂.
(9) Prior to measuring the proliferation status of the cells, an ATP standard curve is performed in exactly the same manner as described in the HALO-96 MeC manual for assay kits. This ATP standard curve allowed the non-standardized Relative Luminescence Units (RLU) to be automatically converted into standardized ATP concentrations (μM).
(10) Each plate of cultured cells is processed by adding 0.1 ml of HALO Monitoring Reagent to each well. Processing is performed using a Beckman Coulter BioMek 2000 liquid handler and an 8-channel pipetting tool, thereby allowing 8-wells to be processed at the same time. The HALO Monitoring Reagent contains a lysis reagent, which lyses the cells to release intracellular ATP. The latter then acts as a limiting substrate for the luciferin/luciferase reagent (containing in the same HALO Monitoring Reagent). After addition and mixing of the well contents, the plate is left to stand at room temperature for 10 minute prior to measuring the luminescence in a LMax plate luminometer (Molecular Devices). The mean, standard deviation and percent coefficient of variation for both the RLU and calculated ATP values are determined.

### Results

i-ATP Assay for CD133⁺ Stem Cell Potency and Proliferative Ability

Stem cell potency may be determined by plotting the mean ATP concentration per well against the number of cells plated. A minimum acceptance criteria may be any suitable level, for example about 1×10⁻⁵ with a potency ratio (PR) of 0.5 (HemoGenix; FIG. 1).

The hematopoietic stem cell potency of cryopreserved CD133¹ cells were assessed using the Hemogenix i-ATP assay prior to transplantation of the cells into NOD/SCID mice. When CD133⁺ HSC GEMM and CD133⁺ HPP were tested for ATP production in this assay, the slopes of the cell dose-responses for both GEMM and HPP were 2.82×10⁻⁴ and 3.52×10⁻⁴ respectively (FIG. 2). These potency measures are greater than the minimum acceptance criteria established by Hemogenix (1×10⁻⁵).

### Methylcellulose Assay for Colony Forming Potential of CD34⁺/CD133⁻ Cells

The methylcellulose colony forming assay can be used to measure the progenitor capacity of a hematopoietic cell population and also provides a measure of engraftment ability for bone marrow transplants.

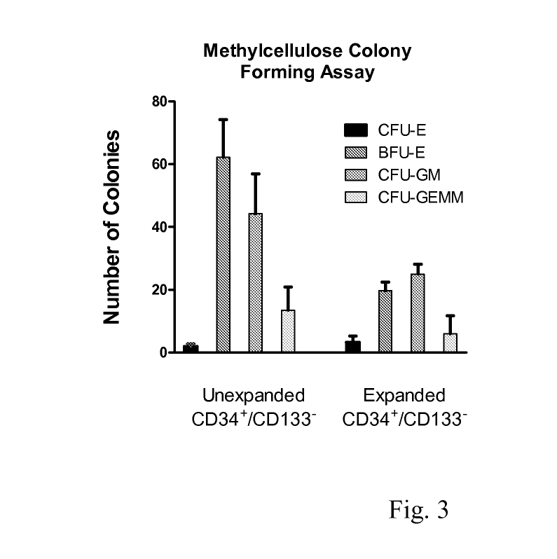

To assess the engraftment potential of unexpanded and expanded CD34⁺/CD133⁻ cells, the number of colonies produced was determined. Both unexpanded and expanded CD34⁺/CD133⁻ cells were able to form the complete repertoire of mature circulatory blood cells (FIG. 3). In one study, the total expansion of cells was about 30-fold (range 10-500 fold). The total number of colonies appearing from 1,000 cells in the unexpanded population was 122, whereas 55 were observed in the expanded population. Hence, the actual fold increase in the numbers of cells giving rise to progenitors in this expansion was 30×(55/122)=13.5 post expansion.

### Engraftment of a 20:80 Mixture of CD133⁺ Cells: CD34⁺/CD133⁻ Cells in NOD/SCID Mice Injected IV or IBM

To compare the effect of the route of administration, a 20:80 mixture of unexpanded and expanded cells, respectively, was administered either intravenously or directly into the bone marrow via an intra-bone marrow injection.

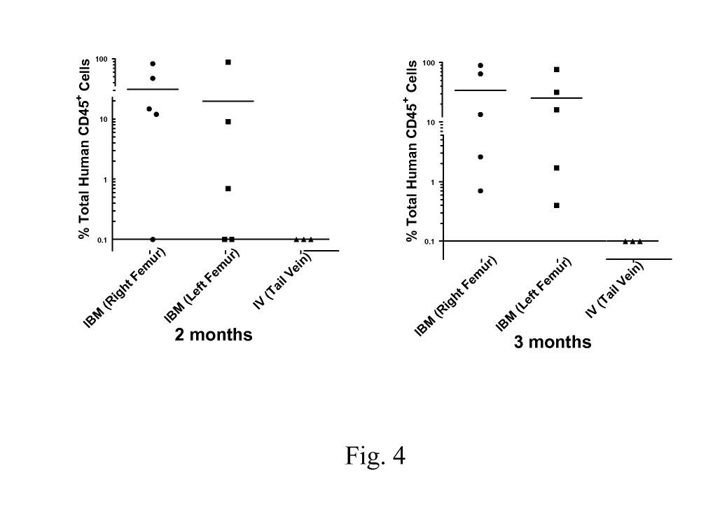

Referring now to FIG. 4, when the cells were administered by an intra-bone marrow injection, the bone marrow from the left femur contained 31±15% human CD45⁺ cells, and the contraleteral femur contained 20±18% human CD45⁺ cells. By contrast, bone marrow harvested from IV injected mice contained less than 0.1% human CD45⁺ cells two months after injection. At 3 months post-intra BM injection, bone marrow cells harvested from the right femur were 34±18% positive for human CD45. Additionally, the left femur of the same mice contained 25±14% human CD45⁺ cells. The same cells injected IV in the tail vein were undetectable in the femoral marrow. Thus, the engraftment multiple is significantly enhanced when the cells are administered IBM relative to IV administration.

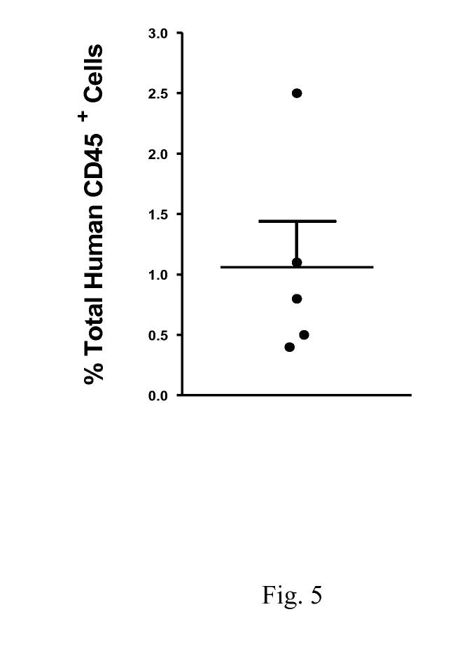

To further confirm that the admixture of CD133⁺: and expanded CD34⁺/CD133⁻ cells had engrafted, peripheral blood was withdrawn from the mice at 3 months post IBM treatment, and there were 1.1±0.4% CD45¹ cells. This robust level of peripheral blood chimerism in this model using 10,000 cells has not been previously reported, and confirms the enhanced efficiency and improvement that is observed with this aspect of the invention (see FIG. 5).

### Serial IV Transplant of CD34⁺ Cells

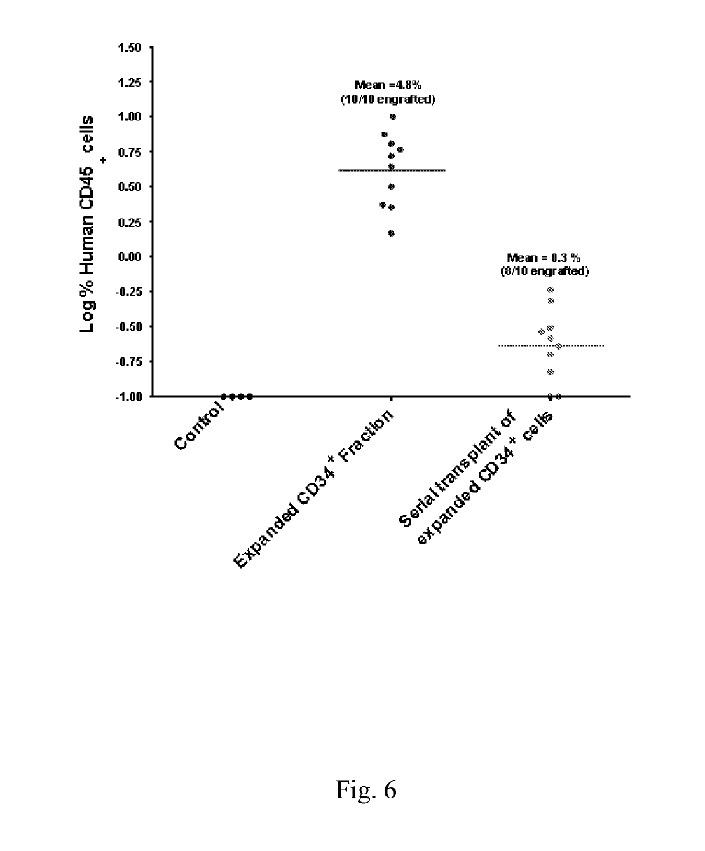

To determine if any true HSC stem cells or SCID Repopulating Cells (SRCs) would survive an expansion protocol, engraftment ability was compared between CD34⁺ expanded cells (Control, FIG. 6) and expanded CD34⁺ cells that were re-purified on CD34⁺ microbeads after seven days of expansion (Expanded CD34⁻ Fraction, FIG. 6). After two months, the percent chimerism from expanded CD34⁻ cells injected IV in the bone marrow was very low (Control, FIG. 6). However, when the expanded CD34⁺ cells were re-selected on the CD34⁺ marker, and injected I.V., ˜4.8% chimerism was observed as measured by human CD45⁺ cells in the marrow (Expanded CD34⁺ Fraction, FIG. 6).

To confirm the ability of expanded CD34⁺ cells to engraft, bone marrow cells harvested after two months were re-administered intravenously into a second NOD/SCID group. After the second transfer, 0.3% chimerism was observed (Serial Transplant of expanded CD34⁻ cells, FIG. 6). These results demonstrate that expansion of the CD34⁺ population allows for the preservation of HSCs. Because of the low frequency of engraftment by the I.V. route, the CD34⁺ cells have to be enriched in order to observe the SRC population in the expanded cells.

### Therapeutic Treatments

Another aspect of the present invention relates to improved therapeutic use of the cells, methods, and/or compositions of the invention. For example, cells, admixtures thereof, and compositions prepared by the methods described herein are useful for treatment of various disorders related to deficiencies in hematopoiesis caused by disease, condition, or myeloablative treatments. As used herein, “treatment” refers to therapeutic or prophylactic treatment. Treatment encompasses administration of the cells of the invention and admixtures thereof in an appropriate form prior to the onset of disease symptoms and/or after clinical manifestations, or other manifestations of the disease or condition to reduce disease severity, halt disease progression, or eliminate the disease. Prevention of the disease includes prolonging or delaying the onset of symptoms of the disorder or disease, preferably in a subject with increased susceptibility to the disorder.

Diseases and/or conditions suitable for treatment with the cells and cell admixtures of the invention include malignant diseases and non-malignant diseases. Malignant diseases include acute lymphocytic leukemia, acute myelocytic leukemia, Juvenile chronic myelogenous leukemia, Chronic myelogeneous leukemia, neuroblasoma, myelodysplatic syndrome. Exemplary non-malignant diseases include Fanconi anemia, Idiopathic aplastic anemia, Thalassemia, Sickle cell anemia, Amegakaryocytic thrombocytopenia, Kostman syndrome, Blackfan-Diamond syndrome, Severe combined immunodeficiency, X-linked lymphocproliferative syndrome, Wiskoff Aldrich syndrome, Hurler syndrome, Hunter syndrome, Gunther disease, Osteopetrosis, Globoid cell leukodystrophy, adrenoleukodystrophy, and Lesch-Nyhan syndrome. Other conditions include neutropenia, a condition characterized by decrease in the amount of circulating neutrophils, and thromobocytopenia, a condition characterized by less than normal levels of platelets in the peripheral blood. Both conditions may be a result of acquired or inherited disorder.

Defective hematopoietic stem cell development is known to occur in diseases manifesting low neutrophil count such as, but not limited to, reticular dysgenesis, Fanconi's anemia, Chediak-Higashi syndrome, and cyclic neutropenia. For thrombocytopenia, low platelet levels are manifested in conditions such as Wiskott-Aldrich Syndrome, thrombocytopenia with absent radii (TAR), and systemic lupus erythematosus. Acquired forms of neutropenia and thrombocytopenia occur under similar circumstances, such as with hematological malignancies, vitamin deficiency, exposure to ionizing radiation, viral infections (e.g., mononucleosis, CMV, HIV, etc.), and following treatment with various cytotoxic drugs.

The cells and admixtures thereof of the invention may be used prophylactically to reduce the occurrence of diseases such as neutropenia and thrombocytopenia, and their associated complications, particularly to lessen infection by opportunistic pathogens in patients that have been treated with myeloablative agents or have undergone HSC transplantation. In the transplant setting, myeloid cells can be used concurrently or subsequent to stem cell transplantation until the recipients' own HSCs or transplanted HSCs begin to restore hematopoiesis and raise neutrophil and platelet levels sufficiently. Infusion of myeloid progenitor cells increases the number of neutrophils and megakaryocytes in the treated subject, thereby providing temporary but needed protection during the neutropenic or thrombocytopenic period. Use of myeloid progenitor cell populations, as opposed to more differentiated neutrophils and platelets, provides for longer lasting protection because of the temporary engraftment of myeloid progenitors and their differentiation in vivo.

The amount or dosage of cells needed for achieving a therapeutic-effect will be determined empirically in accordance with conventional medical procedures for the particular purpose or condition. Generally, for administering cells for therapeutic purposes, cells are given at a pharmacologically effective dose. By “pharmacologically effective amount” or “pharmacologically effective dose” is meant an amount sufficient to produce the desired physiological effect, or amount capable of achieving the desired result, particularly for treating the disorder or disease condition, including reducing or eliminating one or more symptoms or manifestations of the disorder or disease. As an illustration, administration of cells to a patient suffering from neutropenia provides therapeutic benefit not only when the underlying condition is eradicated or ameliorated, but also when the patient experiences a decrease in the severity or duration of the symptoms associated with the disease. Therapeutic benefit also includes halting or slowing the progression of the underlying disease or disorder, regardless of whether improvement is realized.

In one embodiment of this aspect of the invention, cells for administration to a patient include admixtures and/or compositions of purified, expanded CD34⁺ cell populations and purified, unexpanded CD133⁺ cell populations as disclosed herein. Expanded cells may be derived from a single subject, where the cells are autologous or allogeneic to the recipient.

In one embodiment, the expanded and unexpanded cells are cryopreserved and stored frozen. Prior to administration to a patient the frozen cell populations are thawed, mixed in appropriate ratios as described herein and administered to the patient. Preferably the step of admixing the expanded and unexpanded cell populations occurs prior to administration to a patient, for example, immediately prior to, or within about 30 min to about 2-4 hours of administration.

It is to be understood that cells isolated directly from a donor subject without expansion in culture may be used for the same therapeutic purposes as the expanded cells. Preferably, the isolated cells are a substantially pure population of cells. Unexpanded cells may be autologous, where the cells to be infused are obtained from the recipient, such as before treatment with cytoablative agents. In another embodiment, the unexpanded cells are allogeneic to the recipient, where the cells have a complete match, or partial or full mismatch with the MHC of the recipient. The isolated unexpanded cells are preferably obtained from different donors to provide a mixture of allogeneic MNCs.

Transplantation of cells into an appropriate host is accomplished by any suitable method known to the skilled practitioner. For example, administration may be by intravenous infusion or more preferably by intra-bone marrow injection. The number of cells transfused will take into consideration factors such as sex, age, weight, the type of disease or disorder, stage of the disorder, the percentage of the desired cells in the cell population (i.e. purity of cell population), and the cell number needed to produce a therapeutic benefit. Generally, the number of expanded cells infused may be from about 1×10⁴ to about 1×10⁵ cells/kg, from about 1×10⁵ to about 10×10⁶ cells/kg, preferably about 1×10⁶ cells to about 5×10⁶ cells/kg of bodyweight or more as necessary. In one embodiment of the invention, the cells are mixed with any pharmaceutically acceptable carrier, for example, buffered saline at about 1×10⁹ to about 5×10⁹ cells. Cells can be administered in one infusion, or through successive infusions over a defined time period sufficient to generate a therapeutic effect. Different populations of cells may be infused when treatment involves successive infusions.

### Adjunctive Treatments

A variety of adjunctive treatments may be used in combination with the cells of the invention. For example, other agents and compounds that enhance the therapeutic effect of the infused cells, or treat complications may be included. In one aspect, the adjunctive treatments include, but are not limited to, anti-fungal agents, anti-bacterial agents, and anti-viral agents.

In a further embodiment, the adjunctively-administered agent is a cytokine or growth factor that enhances differentiation and mobilization of terminally differentiated myeloid cells, particularly granulocytes, macrophages, megakaryocytes and erythroid cells. For enhancing granulocyte development, the cytokines C-CSF and GM-CSF may be used. G-CSF is effective in accelerating engraftment and production of neutrophils in HSC transplantation. In another embodiment, the cytokine or growth factor is thrombopoietin (TPO). Administration of TPO enhances engraftment of transplanted progenitor cells and promotes development of megakaryocytes and platelets (Fox, N et al., J. Clin. Invest. 110:389-394 (2002); Akahori, H. et al., Stem Cells 14(6):678-689 (1996)).

A variety of vehicles and excipients and routes of administration may be used for adjunctive therapy, as will be apparent to the skilled artisan. Representative formulation technology is taught in, inter alia, Remington: The Science and Practice of Pharmacy, 19th Ed., Mack Publishing Co., Easton, Pa. (1995) and Handbook of Pharmaceutical Excipients, 3rd Ed, Kibbe, A. H. ed., Washington D.C., American Pharmaceutical Association (2000); hereby incorporated by reference in their entirety.

In one aspect, a pharmaceutical composition of the invention will comprise a pharmaceutically acceptable carrier and a pharmacologically effective amount of the cells, composition, compounds, or mixtures thereof of the invention. The pharmaceutical composition may be formulated as solutions or suspensions, or other formulations known in the art.

A pharmaceutical composition of the invention may include one or more buffers (e.g., neutral buffered saline or phosphate buffered saline), carbohydrates (e.g., glucose, mannose, sucrose or dextrans), mannitol, proteins, polypeptides or amino acids such as glycine, antioxidants (e.g., ascorbic acid, sodium metabisulfite, butylated hydroxytoluene, butylated hydroxyanisole, etc.), bacteriostats, chelating agents such as EDTA or glutathione, solutes that render the formulation isotonic, hypotonic or weakly hypertonic with the blood of a recipient, suspending agents, thickening agents, preservatives, as appropriate.

For parenteral administration, the compositions can be administered as injectable dosages of a solution or suspension in a physiologically acceptable diluent with a pharmaceutical carrier that can be a sterile liquid such as sterile pyrogen free water, oils, saline, glycerol, polyethylene glycol or ethanol.

Methods and/or routes of administration include but are not limited to, intravenously and direct injection to specified organs such as e.g., spleen or bone marrow, with intra-bone marrow injection being preferred. Administration of the pharmaceutical compositions of the invention may be through a single route or concurrently by several routes.

The compositions may be administered once per day, a few or several times per day, or even multiple times per day, depending upon, among other things, the indication being treated and the judgment of the prescribing physician.

### Kits

Another aspect of the invention relates to a kit comprising one or more vessels containing expanded CD34⁺ and/or unexpanded CD133⁺ isolated cells, HSCs cytokines and/or growth factors (e.g., G-CSF, GM-CSF, TPO) and/or adjunctive therapeutic compounds. In one embodiment, separate vessels are provide for each cell component, such that admixtures can be prepared prior to therapeutic administration. In another embodiment, expanded and unexpanded HSCs according to the invention are pre-mixed in one or more vessels. A label typically accompanies the kit, and includes any writing or recorded material, which may be electronic or computer readable form (e.g., disk, optical disc, memory chip, or tape) providing instructions or other information for use of the kit.

### Summary

Engraftment efficiency of MNC cells, including cryopreserved cells, is significantly improved by IBM infusion of a mixture of about 20% unexpanded CD133⁺ and about 80% expanded CD34⁺ cells in comparision with I.V. administration.

The HSC content and potency of the unexpanded CD133¹ population can be quantified and standardized by methods described herein. Additionally, the potency of an expanded CD34⁺ fraction to generate precursor colonies of all the blood cells can be quantified and standardized. The repopulating HSC within the mixture of cells injected IBM remain competent, as evidenced by robust engraftment of CD34⁺ cells in the contralateral limb.

It has also been demonstrated that human CD45⁺ cells are present in the blood stream of the IBM injected mice. This level of mature circulating chimeric cells in the bloodstream of HSC injected NOD/Scid mice has not previously been observed. The data indicate that in the human setting, the route of administration and the specific ratio of selected cells is likely to significantly impact the rapidity with which peripheral myeloid cells and platelets are reconstituted. Time to neutophil and platelet engraftment provides two important determinants regarding how long a patient spends in intensive care. In addition, the sooner a patient engrafts, the lower the risk associated with post operative nosocomial infections.

The enhancement of HSC engraftment by IBM administration means that fewer HSC cells are required to successfully engraft a patient. This provides a method for qualifying many more donated units of cord blood for therapeutic transplantation. Moreover, more heavy adults will be able to qualify as suitable recipients for transplant, and more recipient minority races that have substantially lower donor cord blood HSCs, will be able to find a suitable graft. Additionally, the enhancement of HSC engraftment means that a single donated cord blood unit may be adequate for multiple transplants.

The invention has been described with reference to various illustrative embodiments and techniques. However, it should be understood that many variations and modifications as are known in the art may be made while remaining within the scope of the claimed invention. The examples that follow are illustrative and are not intended to be limiting.

### EXAMPLE 1

### Preparation of Human Cord Blood MNC

Using a Syringe-Stopcock-extension unit, equal volumes of Prepacyte®-WBC is added to umbilical cord blood (e.g. equal volume of Prepacyte®-WBC added to whole blood contained in bag). After mixing, plasma is separated from red blood cells by suspending cord blood bag vertically from a plasma extractor or a retort stand with three pronged clamps for 30 minutes. The plasma layer is removed from red blood cells and transferred to 50 mL, sterile conical flasks. The MNC cell fraction is pelleted at 400×g for 7-10 minutes, at room temperature. Supernatant fluid is aspirated from the total nucleated cell (TNC) pellet. Pellets are resuspended in 1 mL PBS containing 0.5% BSA and 2 mM EDTA (PBE). Cell number and viability are assessed using a hemacytometer or Guava Viacount reagents.

### EXAMPLE 2

### Immuno-Magentic MACS Separation of CD34⁺ Cells from TNC Fraction

A TNC pellet is resuspended in 300 μL PBE per 1×10⁸ TNC and 100

μL FcR block added along with 100 μL CD34⁺ microbeads. The mixture is gently swirled and allowed to stand for 30 minutes in a refrigerator. Thereafter the cells are washed twice with 30 mL of PBE and the cells pelleted at 300×g for 10 minutes at 2-8° C. The cell pellet is resuspended in 1 mL PBE per 1×10⁸ TNC. AutoMACS™ Pro is used to purify the CD34 cells labeled with the CD34¹ immunomagnetic beads as follows. CD34⁻ cells are pelleted at 300×g for 7-10 minutes at 2-8° C. and resuspended in 0.5-1.0 mL of HSC re-suspension media (EndGenitor). Cell number and viability are assessed using Viacount® reagents and Guava® EasyCyte or by using a hemacytometer and trypan blue. Purity of the CD34⁻ cells is confirmed by labeling isolated cells with fluorescently labeled, anti-human CD34⁻ antibody and Guava® EasyCyte. Purified cells are cryopreserved and stored in liquid nitrogen.

### EXAMPLE 3

### Immuno-Magentic MACS Separation of CD133⁺ Cells from TNC Fraction

Approximately 1×10⁸ TNC is resuspended in 300 μL PBE. About 100 μL FcR block is added to 1×10⁸ TNC and incubated at 2-8° C. for 10 minutes. Thereafter, about 50 μL CD133⁺ microbeads are added and incubated for 20 minutes in 2-8° C. refrigerator. The mixture is then washed with 30 mL of PBE, pelleted at 300×g for 10 minutes at 2-8° C., and resuspended in 1 mL PBE. CD133+ cells are purified using the AutoMACS™ Pro to isolate the CD133⁺ cells labeled with the CD133⁺ immunomagnetic beads. Following purification of CD133⁺ cells on magnetic columns, CD133⁺ cells are pelleted at 300×g for 7-10 minutes at 2-8° C. and resuspended in 0.5-1.0 mL of EGT HSC re-suspension media. Cell number and viability are assessed using Viacount® reagents and Guava® EasyCyte (or use hemacytometer and trypan blue). Purity of CD133¹ cells is confirmed by labeling isolated cells with fluorescently labeled, anti-human CD133⁺ antibody and Guava® EasyCyte. Purified CD 133+ cells are cryopreserved and stored in liquid nitrogen freezer.

### EXAMPLE 4

### Cryopreservation of Myeloid Progenitor Cells

Cells are concentrated, suspended in a medium supplemented with a cryoprotectant and/or stabilizer, frozen and stored at a temperature of 0° C. or less. The cells are mixed with a suitable cryoprotectant such as dimethyl sulfoxide (DMSO) or glycerol. For example, a freezing medium comprises DMSO from about 5-10%, 10-90% serum albumin, and 50-90% culture medium. The cells can be stored in any stabilizer known in the art. For example, the stabilizer may be methyl cellulose or serum.

### EXAMPLE 5

### Mouse Intra-Bone Marrow Transplant and Bm/Peripheral Blood Harvest

An admixture of expanded CD34⁺ HSCs and unexpanded CD133⁺ cells is prepared as follows. Frozen umbilical cord blood (UCB)-derived expanded CD34⁺ hematopoietic stem cells are thawed and mixed with frozen and thawed CD133⁺ cells in a ratio of about 80:20 respectively. Mixed cells may be resuspended in a suitable injection buffer at a concentrations of about 8×10⁹ cells/240 μL of DPBS⁺0.5% BSA. One group, the vehicle control, receives only buffer injections. Cells are injected directly into the bone marrow of the right leg of each mouse at a concentration of about 1×10⁴ cells/30μL. At endpoint (2-3 months), mice are sacrificed and bone marrow and plasma are collected. Plasma is collected via a tail vein and the femur and tibia are removed from both hind limbs. Connective and/or muscle tissue is removed using sterile gauze pads. Bones from each leg are placed in individual sterile tissue culture dishes containing 5 mL RPMI 1640 modified tissue culture media supplemented with 10% Fetal Bovine Serum (ATCC). After removing distal ends of bones all marrow is flushed into media using a sterile 5 ml syringe and sterile 0.5-in 27 g needle for processing and analysis. Samples undergo red cell reduction (R&D Systems) by lysis and are then washed and counted.

### EXAMPLE 6

### Methylcellulose Colony Forming Cell Assay (CFC)

Colony formation assays of HSCs are done to assess the functional capacity of progenitor cell subsets for hematopoiesis regeneration in bone marrow transplantation. CFC assays are performed using the Complete MethoCult® assay (StemCell Technologies) according to the manufacturers recommended procedure. Briefly, thawed or fresh HSCs are cultured in duplicate in complete methylcellulose media at a concentration of about 500 cells/plate. Plates are incubated in a humidified, 37° C. incubator at 5% CO₂ for 14-16 days. Total erythroid, granulopoietic, and multi-lineage colonies are enumerated by light microscopy.

### EXAMPLE 7

### Ex Vivo Expansion of CD34+/CD133+ Cells

Umbilical cord blood derived CD34+/CD133+ cells are aliquoted into six-well culture dishes at 1×10⁶ cells per well. The cells are allowed to proliferate for 7 days in a humidified incubator at 37° C. and 5% CO₂ in standard HSC culture medium containing 100 ng/ml SCF, FLT-3, and TPO (EndGenitor Technologies). The expanded cells are harvested, washed with PBS containing 0.5% serum albumin and 2 mM EDTA, and counted. Fold-expansion is calculated as total viable expanded cells less viable cells seeded divided by viable cells seeded.

1. A method for improving hematopoietic stem cell engraftment in a patient in need thereof following HSC transplantation comprising administration of a mixture of purified expanded hematopoietic stem cells and purified unexpanded hematopoietic stem cells wherein said expanded cells are CD34⁺/CD133⁻ and said unexpanded cells are CD133⁺

2. The method of claim 1 wherein said cells are cryopreserved and wherein said mixture has a TNC in a range of about 1×10⁸ to about 8×10⁸.

3. The method of claim 2 wherein said cells are isolated from human umbilical cord blood.

4. The method of claim 3 wherein said mixture comprises a ratio of expanded cells to unexpanded cells in a range of about 90:10 to about 60:40, said method further comprising the step of determining potency of said unexpanded cells prior to administration.

5. (canceled)

6. (canceled)

7. The method of claim 4 wherein said administration is by IV injection or intra-bone marrow injection.

8. The method of claim 7 wherein said expanded cells are re-purified after expansion by selection for the CD34⁺ marker.

9. The method of claim 7 wherein said administration is by intra-bone marrow injection.

10. (canceled)

11. (canceled)

12. The method of claim 7 wherein said patient experiences a clinical benefit selected from the group consisting of reduced time to myeloid replacement, reduced time to neutrophil engraftment, and reduced time to platelet engraftment.

13. A process for improving transplant potential of cord blood comprising the steps of:
a) purifying a CD34⁺/CD133− subset from cord blood;
b) expanding the CD34+/CD133− subset of step (a) about 5-fold to 500-fold to yield an expanded CD34+/CD133− subset;
c) purifying a CD133⁺ subset from cord blood wherein said CD133+ subset is unexpanded;
d) cryopreserving the subsets from step b) and step c); and
e) admixing said expanded CD34⁺/CD133− subset and said purified unexpanded CD133− subset in a ratio of about 90:10 to about 60:40, and wherein said purified CD34⁺/CD133− and CD133⁺ subsets meet a threshold potency requirement.

14. The process of claim 13 wherein the TNC of a unit of said cord blood is in a range of about 1×10⁸ to about 8×10⁸.

15. The process of claim 14 wherein the potency for said CD133⁺ cells is determined by measuring i-ATP levels.

16. A therapeutic composition comprising expanded and unexpanded HSCs from human cord blood in a pharmaceutically acceptable carrier wherein said expanded cells are CD34⁺/CD133⁻ and said unexpanded cells are CD133⁺, and wherein the ratio of said expanded to unexpanded cells is in a range of about 90:10 to about 60:40 and wherein said unexpanded HSCs possess a threshold potency.

17. The composition of claim 16 wherein said potency is based on measuring i-ATP levels.

18. (canceled)

19. (canceled)

20. A kit for hematopoietic stem cell transplantation comprising at least one vessel containing a composition of claim 16.

21. (canceled)

22. (canceled)

23. A method for treating a patient suffering from impaired hematopoiesis comprising administering to said patient a therapeutic composition according to claim 16.

24. The method of claim 23 wherein said patient is undergoing hematopoietic stem cell transplantation.

25. The method of claim 23 wherein said patient is suffering from a malignant or non-malignant disease.

26. The method of claim 25 wherein said malignant disease is selected from the group consisting of acute lymphocytic leukemia, acute myelocytic leukemia, Juvenile chronic myelogenous leukemia, Chronic myelogeneous leukemia, neuroblasoma, myelodysplatic syndrome.

27. The method of claim 25 wherein said non-malignant disease is selected from the group consisting of Fanconi anemia, idiopathic aplastic anemia, thalassemia, sickle cell anemia, amegakaryocytic thrombocytopenia, Kostman syndrome, Blackfan-Diamond syndrome, severe combined immunodeficiency, X-linked lymphocproliferative syndrome, Wiskoff Aldrich syndrome, Hurler syndrome, Hunter syndrome, Gunther disease, osteopetrosis, globoid cell leukodystrophy, adrenoleukodystrophy, and Lesch-Nyhan syndrome, neutropenia, and thromobocytopenia.
